(19) United States
(12) Patent Application Publication
(10) Pub. No.: US 2020/0323591 A1
(43) Pub. Date: Oct. 15, 2020

(54) Twin fiber laser system for the treatment of enlarged prostate

(71) Applicant: John B. RANSOME, Fairfield, CT (US)

(72) Inventor: John B. RANSOME, Fairfield, CT (US)

(21) Appl. No.: 16/818,128

(22) Filed: Mar. 13, 2020

Related U.S. Application Data
(60) Provisional application No. 62/819,065, filed on Mar. 15, 2019.

(51) Int. Cl. A61B 18/22; A61B 1/307; A61B 1/00; A61B 1/07; A61B 1/12

(52) U.S. Cl. CPC A61B 18/22; A61B 1/307; A61B 1/00195; A61B 2018/00982; A61B 1/07; A61B 1/126; A61B 1/00188; A61B 1/00135

(57) ABSTRACT

A medical system is provided including a laser module housing comprising a plurality of laser generating engines; a plurality of optical fibers, each configured to be connected to one of the plurality of laser generating engines of the laser module housing and to deliver a laser to a tissue of a patient during a medical procedure; and a resectoscope comprising a sheath configured to receive the plurality of optical fibers and deliver the lasers from the plurality of optical fibers to the tissue of the patient through an opening in the sheath at a first end of the resectoscope.

### CROSS-REFERENCE TO RELATED APPLICATIONS

The present application claims the benefit of U.S. Provisional Patent Application No. 62/819,065, filed Mar. 15, 2019, which is hereby incorporated by reference in its entirety.

### BACKGROUND OF THE INVENTION

Having an enlarged prostate is an annoying problem for older men all over the world. With multiple late night trips to the bathroom, it is a problem that also affects their spouses. Enlarged prostate (Benign Prostatic Hyperplasia or “BPH”) effects 50% of men over the age of 60, and to 90% by age 85.

The prostate is a walnut-sized gland located between the bladder and the penis. The urethra runs through the center of the prostate letting urine flow out of the body. Each day as the prostate grows, that enlargement blocks the flow of urine. Eventually the gland grows big enough to put pressure on the urethra and the bladder must contract more forcefully to push the urine out. Over time the muscles in the bladder wall become stronger, thicker, and overly sensitive, causing a need to urinate frequently. Symptoms of BPH include: a weak or slow urinary stream, a feeling of incomplete bladder emptying, difficulty starting urination, frequent urination/urgency to urinate, getting up frequently at night, a urinary stream that starts and stops, straining to urinate, continued dribbling of urine, and returning to urinate minutes after finishing.

Most men live with an enlarged prostate for months, even years before seeing a doctor. They often seek out an urologist only when they are getting up several times per night and having trouble falling asleep.

When the time comes for a surgical procedure to treat BPH, the urologist has two main modalities to choose from, a TURP (Transurethral Resection of the Prostate) or lasers. TURPS using an electrocautery device make up the majority of market share today. In the TURP procedure, the surgeon uses an electrical cutting loop to remove small pieces of tissue from the interior of the prostate.

While effective, the procedure is known to cause several side effects, including incontinence, impotence, prolonged bleeding and TUR syndrome. This syndrome is caused when too much of the fluid used during the procedure gets absorbed into the bloodstream. Although rare, if left untreated, life-threatening problems can develop such as seizures, shortness of breath, blue skin and comas. Another disadvantage of this procedure is that patients are required to stay overnight in the hospital.

There are disadvantages for the surgeons as well, as there is typically more bleeding to manage during this type of procedure. The surgeon must routinely stop to coagulate the tissue to see what they are doing. When the visibility is clear, the surgeon can only cut in one direction when performing a TURP, and oftentimes has to stop to clear small chunks of tissue from the metal loop. At the end of the procedure, the surgeon needs to remove all the pieces of tissue from the bladder, which takes time. The scope that the surgeon inserts into the penis has a larger diameter which could also cause strictures post-operatively. So while this is currently the fastest way to reduce the size of the enlarged prostate, it is inefficient.

Lasers are the second modality of choice, but the current lasers available for treating BPH are slow and often the procedure needs to be redone much sooner than should be necessary. Side firing lasers used in the prostate are designed to vaporize the tissue instead of cutting the tissue. The “GreenLight” laser is currently the most widely used in the United States and was the first to come out with the application for BPH. The problem with such a laser is that when the laser fires, the beam shoots out like a blow torch, making it difficult for the surgeon to see what he or she is vaporizing. If the surgeon wants to take tissue he or she cannot do so with the side firing laser and visibility is poor because of the blow torch effect when firing the laser. Currently the main issues with lasers are they are slow and inefficient.

### SUMMARY OF THE INVENTION

The present application relates to a much faster surgical laser system for the treatment of enlarged prostate. The system of the present application includes twin laser fibers, using two laser fibers to deliver energy to the tissue instead of one. This makes the laser twice as fast as any other laser available. The system also comprises a disposable resectoscope that guides the fibers into the patient. This new handheld scope allows the surgeon to free up a hand and is 40% lighter than the stainless steel instruments currently in use. This new laser system solves the problems of both the patient and the surgeon by delivering speed, safety and higher efficiency. The patient will benefit due to the quick turnaround time of the procedure. Instead of having to undergo anesthesia for just under an hour, this new laser cuts that time down to 10-15 minutes. This is also an outpatient procedure so the patient can go home the same day.

As for the surgeons, the time savings is a huge benefit, as they can get back to the office to see more patients making their day more efficient. The speed at which the surgeon can remove tissue allows the surgeon to stop bleeding quicker making it safer and eliminating the need to switch over to a TURP procedure. Physicians typically choose the TURP procedure if the patient's prostate is large because current lasers are too slow. The laser system of the present application is as fast, and in most cases faster than a TURP.

The new laser system also saves the hospital and surgery center from buying expensive stainless steel instruments. Each sterile package will contain a fiber and the first disposable laser resectoscope on the market. This eliminates the time needed to sterilize instruments in between patients when there are multiple procedures booked for the day. It also eliminates the possibility of cross contamination from scopes that were not cleaned properly.

The new fiber will be a contact fiber, meaning the surgeon has to touch the tissue in order to vaporize the tissue, which is safer than a firing laser. The surgeon can also make cuts with the laser, in order to take tissues samples, such as for sending to pathology to check for prostate cancer. This cannot easily be done with other lasers.

In accordance with a first aspect of the present application, a medical system is provided comprising: a laser module housing comprising a plurality of laser generating engines; a plurality of optical fibers, each configured to be connected to one of the plurality of laser generating engines of the laser module housing and to deliver a laser to a tissue of a patient during a medical procedure; and a resectoscope comprising a sheath configured to receive the plurality of optical fibers and deliver the lasers from the plurality of optical fibers to the tissue of the patient through an opening in the sheath at a first end of the resectoscope.

The medical system may further comprise a surgical lens configured to be inserted into the resectoscope for visualizing the tissue of the patient. In certain embodiments, the surgical lens is a cystoscope.

In accordance with one or more of the above-described embodiments of the medical system, the sheath of the resectoscope may comprise: a laser passageway configured to receive a portion of the plurality of optical fibers; and a cystoscope passageway configured to receive the cystoscope. The resectoscope may further comprise: a fluid intake port configured to intake a fluid into the sheath of the resectoscope; a first fluid passage in the sheath of the resectoscope configured to deliver fluid from the fluid intake port into the patient; a second fluid passage in the sheath of the resectoscope configured to receive a fluid flow from the patient; and a fluid outlet port configured to provide an outlet from the resectoscope for the fluid flow from the patient.

The resectoscope of the medical system may further comprise a handle, and/or a plunger configured to push the plurality of optical fibers forward in the patient when actuated by a user. In embodiments of the resectoscope, the plunger comprises: a spring configured to bias the plunger away from the handle, and a thumb or finger hole, configured to receive a thumb or finger of the user. In additional or alternative embodiments, the resectoscope further comprises a fiber port at a second end of the resectoscope configured to receive the plurality of optical fibers for providing to the sheath, and the fiber port can be clipped onto the plunger. The resectoscope further comprises a lens entry port at a second end of the resectoscope configured to receive the surgical lens for providing to the sheath. In further additional or alternative embodiments, the resectoscope further comprises a lens adapter connected to lens entry port configured to extend the length of the resectoscope and to receive the surgical lens for providing to the sheath.

In further embodiments of the medical system, the resectoscope is made of a plastic material and is disposable.

In accordance with embodiments of the medical system, the plurality of optical fibers comprises two optical fibers and the plurality of laser generating engines comprises two laser generating engines. Each of the plurality of optical fibers may comprise a first end having a fitting configured to be received in a port of one of the plurality of laser generating engines to receive the laser, and/or each of the plurality optical fibers further comprises a second end configured to deliver the laser to the patient, wherein each of the second ends and the first end of the resectoscope are cut at an angle. Each of the plurality of optical fibers may be contact fibers configured to cut the tissue of the patient during the medical procedure upon contact with the tissue. In certain embodiments of the medical system, a terminating portion of the two optical fibers are affixed together for insertion into the resectoscope.

In accordance with additional or alternative embodiments of the medical system, each of the laser generating engines is a diode engine. The two laser generating engines may also be configured to each provide a laser beam having a different wavelength to the two optical fibers.

In accordance with further aspects of the invention, a laser module housing, the plurality of optical fibers and the resectoscope according to the above-referenced embodiments are provided independent from the medical system and additional elements thereof.

### BRIEF DESCRIPTION OF THE DRAWINGS

FIG. 1A shows a first perspective view of a resectoscope according to an embodiment of the application.

FIG. 1B shows a second perspective view of the resectoscope according to an embodiment of the application.

FIG. 1C shows a side view of the resectoscope according to an embodiment of the application.

FIG. 1D shows a top side view of the resectoscope according to an embodiment of the application.

FIG. 1E shows an end view of the resectoscope according to an embodiment of the application.

FIG. 2A shows a first perspective view of a resectoscope comprising an adapter according to a further embodiment of the application.

FIG. 2B shows a second perspective view of the resectoscope comprising the adapter according to a further embodiment of the application.

FIG. 2C shows a side view of the resectoscope comprising the adapter according to a further embodiment of the application.

FIG. 2D shows a top side view of the resectoscope comprising the adapter according to a further embodiment of the application.

FIG. 2E shows an end view of the resectoscope comprising the adapter according to a further embodiment of the application.

FIG. 3A shows a first view of a sheath of a resectoscope according to a further embodiment of the application.

FIG. 3B shows a second, perspective view of the sheath of a resectoscope according to a further embodiment of the application.

FIG. 3C shows a cross-sectional view of a sheath of a resectoscope according to a further embodiment of the application.

FIG. 4 shows a diagram of a laser module according to an embodiment of the application.

### DETAILED DESCRIPTION OF THE INVENTION

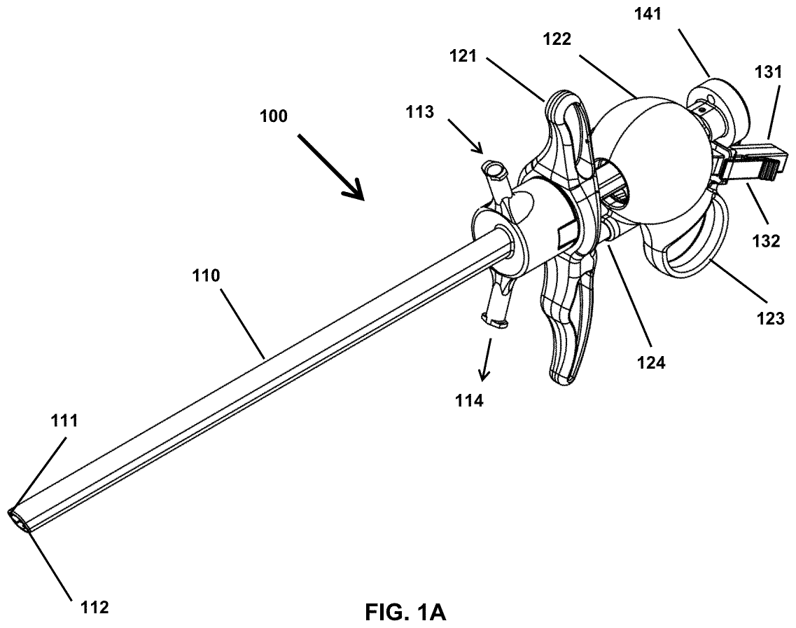

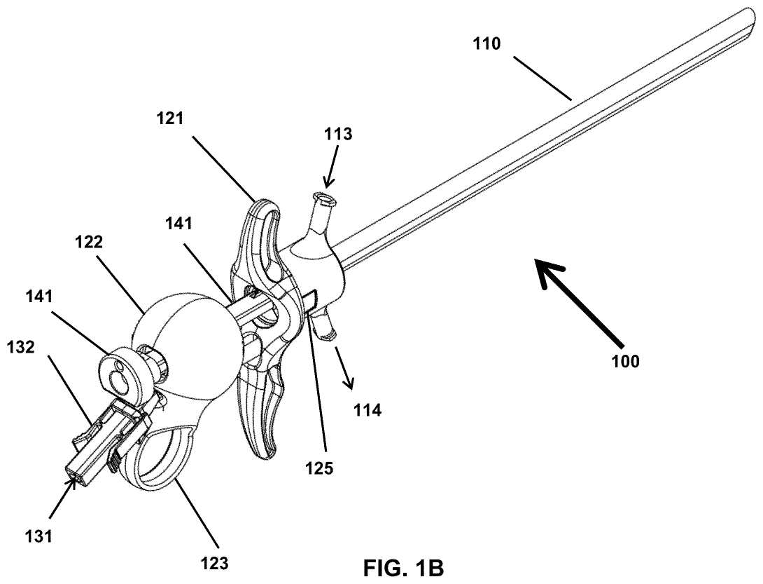

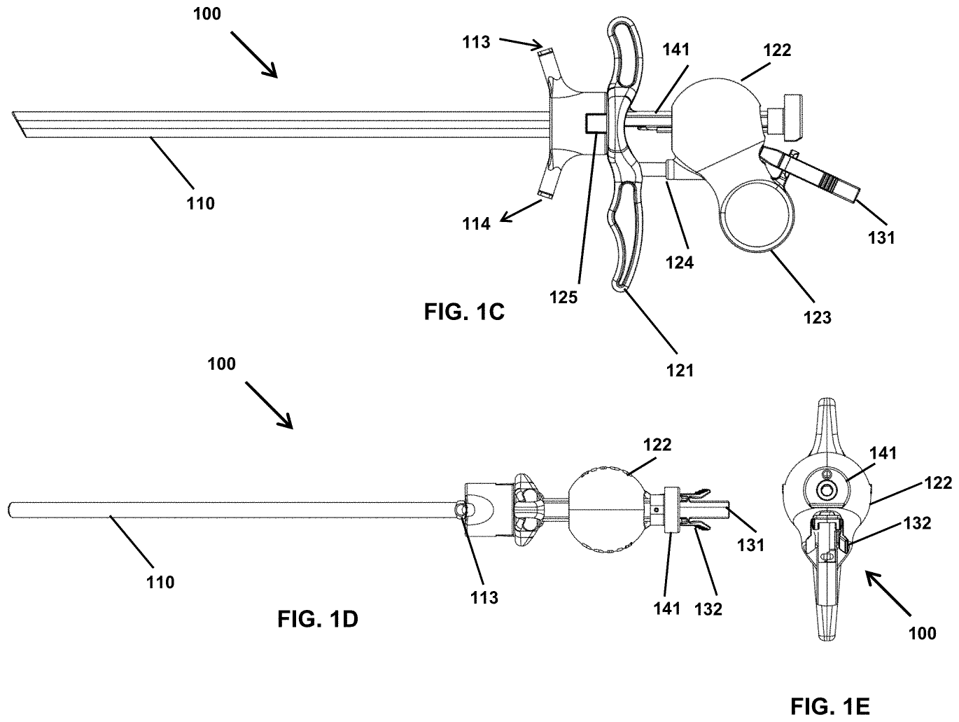

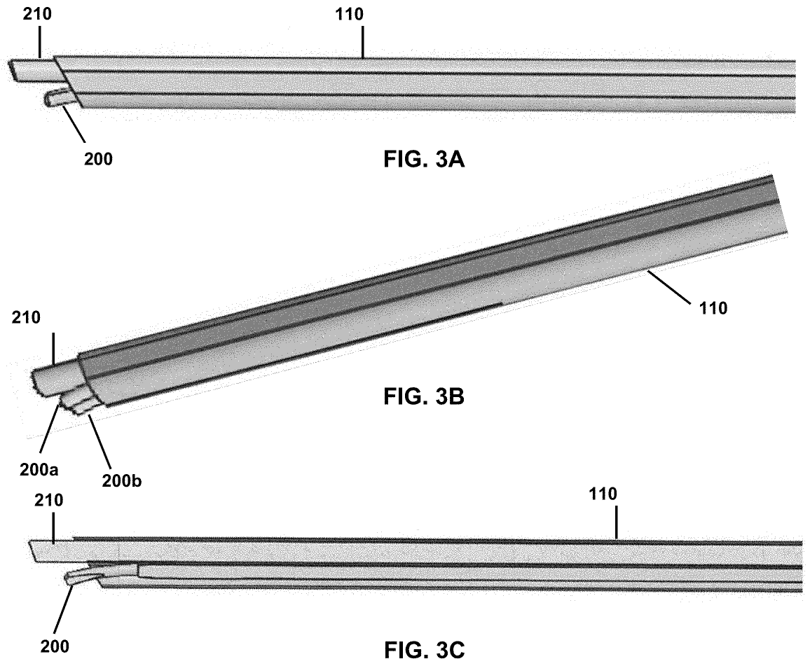

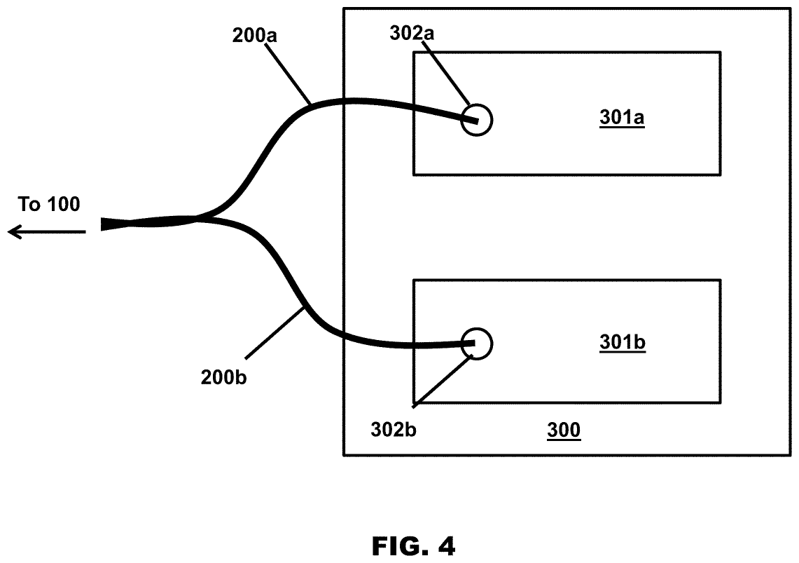

The medical device of the present application will now be described with reference made to FIGS. 1A-4.

In accordance with one embodiment, the medical device or system of the present application may include three separate components, which in combination operate the system as indicated, including a resectoscope 100, twin laser fibers 200 and a laser module housing 300.

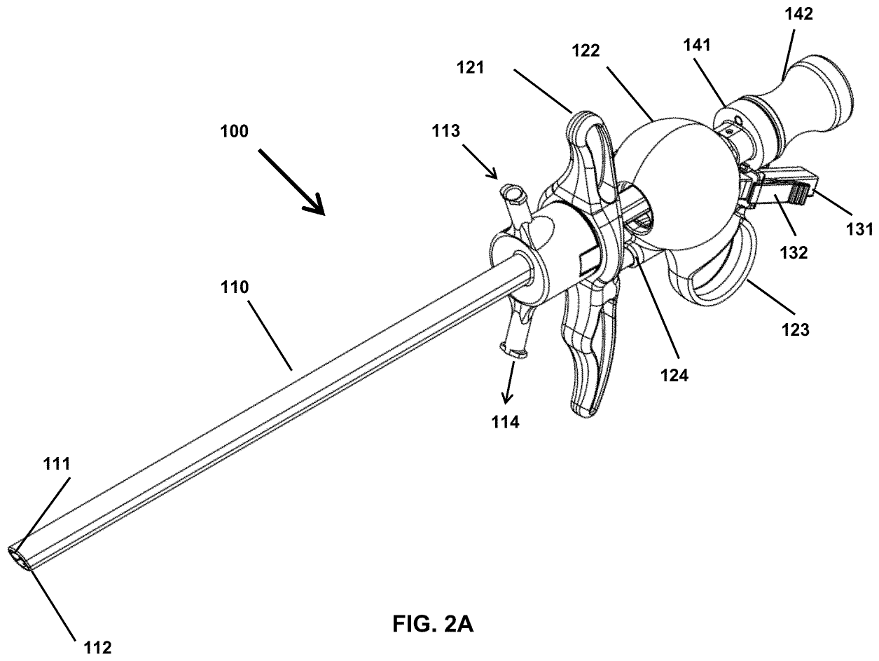

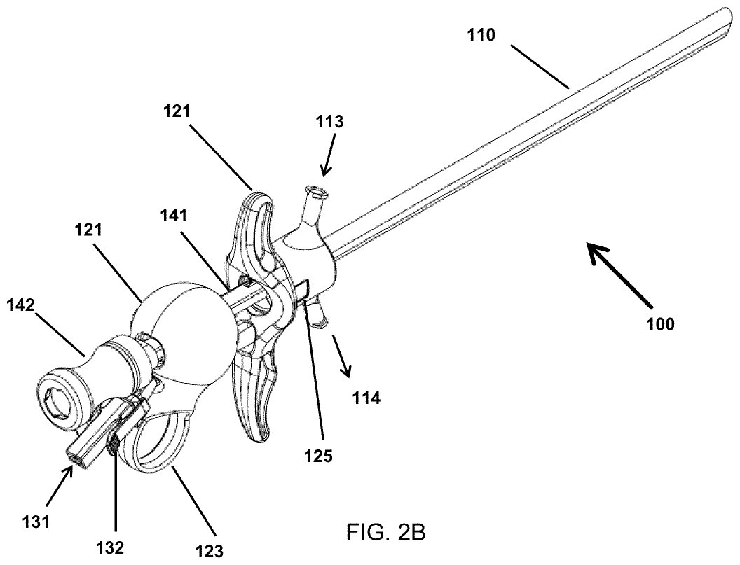

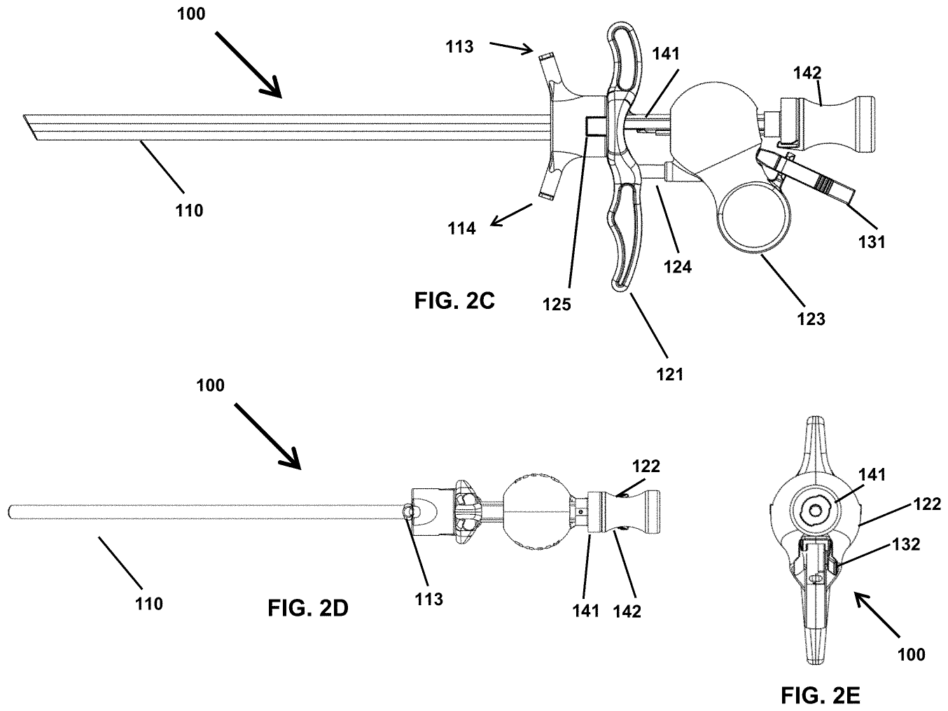

The first component of the system is a resectoscope 100, which holds the laser fibers 200 in place and controls the movement of the fibers 200 inside the patient's body. The resectoscope 100 may be plastic and disposable. Examples of the resectoscope 100 are shown in FIGS. 1A-1E and 2A-2E.

The resectoscope 100 acts as the introducer of saline or other fluid solution into the patient and controls the inflow and outflow of irrigation. The resectoscope 100 comprises a sheath 110, which houses the laser fibers 200 and a cystoscope 210 or other surgical lens for visualization and light. Passages 111, 112 are provided through the sheath 110 for the twin laser fibers 200 and the cystoscope 210. The sheath 110 also comprises sufficient space in the cystoscope or lens passage 111 and fiber passage 112 around the fibers 200 and the cystoscope 210 or other surgical lens, or comprises separate passages, to allow the saline fluid entering the resectoscope 100 through a fluid inlet 113 to pass through the sheath 110 into the patient, and to flow out of the patient and through the sheath 110 to a fluid outlet 114 of the resectoscope 100.

The resectoscope 100 includes a handle 121 and a plunger 122 having a thumb holder 123 to allow the surgeon to manipulate the resectoscope 100. In one embodiment shown in the Figures, the plunger 122 is ball shaped, with a thumb holder 123 attached to the ball 122. Within the plunger 122, a spring (not shown) is affixed to a support beam 124, which biases the plunger 122 away from the sheath 110. The segment of the resectoscope 100 comprising the handle 121 and plunger 122 can be removably docked to sheath 110 of the resectoscope 100, in case the two parts need to be disconnected for cleaning. The handle 121 and plunger 122 are separable as a unit from the sheath 110 and the fluid inlet/outlet 113/114. The docking 125 can include a tab, snap fit, threaded fittings, male-female connectors, or any other suitable locking mechanism for connecting the sheath 110 and fluid inlet/outlet 113/114 to the handle 121 and plunger 122.

The fibers 200 are attached and inserted into the sheath 110 through a port 131, which may be attached to the ball 122 or the thumb holder 123 of the plunger 122. The fiber port 131 may include one or more clips 132 to secure the fiber port 131 to the resectoscope 100.

The resectoscope 100 can be used with multiple types of surgical lenses or cystoscopes. The end of the resectoscope 100 opposite the end of the sheath 110 comprises an entry way 141 for the cystoscope 210, which passes through the ball of the plunger 122 and through the sheath 110. Embodiments of the resectoscope 100 as shown in FIGS. 1A-1E, for example, can utilize a cystoscope 210, such as a cystoscope 210 manufactured by OLYMPUS. A cystoscope adapter 142 can also be provided that is attached to the cystoscope entry 141 at the end of the resectoscope 100, as shown for example in FIGS. 2A-2E. The adapter 142 enables the same resectoscope 100 to be used with a cystoscope 210 having a different length, such as a Storz-type cystoscope 210, which may be longer. As a result, the adapter 142 allows a surgeon or physician to maintain a single type or size of resectoscope 100 that can be adapted for use with multiple cystoscopes 210, rather than requiring multiple types and sizes of resectoscopes 100 to be kept on hand.

A second component of the system includes the twin laser fibers 200a, 200b that carry the energy created by the lasers 301a, 301b to the selected tissue being vaporized. In one embodiment of the system, the fibers 200a, 200b are made from silica glass and separately connect to the laser module housing 300 using plastic or metal fittings at one of two ports 302a, 302b on the front of the laser module housing 300. In embodiments of the system, approximately the last two feet of the fibers 200a, 200b can be fused together for entry into the resectoscope 100. In certain embodiments, the fibers 200a, 200b are covered with a special polymer that has a luminescent coating, making the fibers 200a, 200b more visible during the surgical procedure. The tips of the fibers 200a, 200b can be formed with a slight downward angle, rather than a flat end, in order to cut and vaporize the tissue more efficiently.

A further component of the system is a laser module housing 300 which houses the two engines 301a, 301b, one for each of the two lasers supplying energy to the twin laser fibers 200a, 200b. For example, the module housing 300 may include two diode or thulium engines, one engine 301a continuously using 980 nm wavelength and a second engine 301b continuously using 1480 nm wavelength. Light of other wavelengths may also be used and generated, and other laser systems may be used. The power from each laser engine 301a, 301b can range from five to two hundred watts. In certain embodiments, the laser module housing 300 comprises a touchscreen display (not shown) that can be used to operate the system, and/or a foot pedal (not shown) where the physician can control power settings for each engine 301a, 301b and fiber 200a, 200b, and the ready standby mode. The laser module housing 300 may be mounted on a stand, including for example a mobile portable stand with wheels, which may also be collapsible. The module 300 housing can be made of plastic or other materials and can also include a compartment for laser safety glasses.

It is noted that the resectoscope 100, laser fibers 200 and laser module housing 300 described above can be provided separately or used with other elements, and are not limited to use only in a combination comprising all three components as described above.

While there have been shown and described and pointed out fundamental novel features of the invention as applied to preferred embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice.

What is claimed:

1. A medical system comprising:
a laser module housing comprising a plurality of laser generating engines;
a plurality of optical fibers, each configured to be connected to one of the plurality of laser generating engines of the laser module housing and to deliver a laser to a tissue of a patient during a medical procedure; and
a resectoscope comprising a sheath configured to receive the plurality of optical fibers and deliver the lasers from the plurality of optical fibers to the tissue of the patient through an opening in the sheath at a first end of the resectoscope.

2. The medical system according to claim 1, further comprising a surgical lens configured to be inserted into the resectoscope for visualizing the tissue of the patient.

3. The medical system according to claim 2, wherein the surgical lens is a cystoscope.

4. The medical system according to claim 3, wherein the sheath of the resectoscope comprises:
a laser passageway configured to receive a portion of the plurality of optical fibers; and
a cystoscope passageway configured to receive the cystoscope.

5. The medical system according to claim 1, wherein the resectoscope further comprises:
a fluid intake port configured to intake a fluid into the sheath of the resectoscope;
a first fluid passage in the sheath of the resectoscope configured to deliver fluid from the fluid intake port into the patient;
a second fluid passage in the sheath of the resectoscope configured to receive a fluid flow from the patient; and
a fluid outlet port configured to provide an outlet from the resectoscope for the fluid flow from the patient.

6. The medical system according to claim 2, wherein the resectoscope further comprises a handle.

7. The medical system according to claim 6, wherein the resectoscope further comprises a plunger configured to push the plurality of optical fibers forward in the patient when actuated by a user.

8. The medical system according to claim 7, wherein the plunger comprises:
a spring configured to bias the plunger away from the handle, and
a thumb or finger hole, configured to receive a thumb or finger of the user.

9. The medical system according to claim 7, wherein the resectoscope further comprises a fiber port at a second end of the resectoscope configured to receive the plurality of optical fibers for providing to the sheath.

10. The medical system according to claim 9, wherein the fiber port is clipped onto the plunger.

11. The medical system according to claim 7, wherein the resectoscope further comprises a lens entry port at a second end of the resectoscope configured to receive the surgical lens for providing to the sheath.

12. The medical system according to claim 11, wherein the resectoscope further comprises a lens adapter connected to lens entry port configured to extend the length of the resectoscope and to receive the surgical lens for providing to the sheath.

13. The medical system according to claim 1, wherein the resectoscope is made of a plastic material and is disposable.

14. The medical system according to claim 1, wherein the plurality of optical fibers comprises two optical fibers and the plurality of laser generating engines comprises two laser generating engines.

15. The medical system according to claim 1, wherein each of the plurality of optical fibers comprises a first end having a fitting configured to be received in a port of one of the plurality of laser generating engines to receive the laser.

16. The medical system according to claim 15, wherein each of the plurality optical fibers further comprises a second end configured to deliver the laser to the patient, wherein each of the second ends and the first end of the resectoscope are cut at an angle.

17. The medical system according to claim 16, wherein each of the plurality of optical fibers are contact fibers configured to cut the tissue of the patient during the medical procedure upon contact with the tissue.

18. The medical system according to claim 14, wherein a terminating portion of the two optical fibers are affixed together for insertion into the resectoscope.

19. The medical system according to claim 14, wherein each of the laser generating engines is a diode engine.

20. The medical system according to claim 14, wherein the two laser generating engines is configured to provide a laser beam having a different wavelength to the two optical fibers.
